(19) United States
(12) Patent Application Publication
(10) Pub. No.: US 2012/0022454 A1
(43) Pub. Date: Jan. 26, 2012

(54) CATHETER TIP ASSEMBLY AND METHOD OF USING THE SAME

(75) Inventors: James Wall, San Francisco, CA (US); Eric Willis, Santa Cruz, CA (US)

(73) Assignee: INSITE MEDICAL TECHNOLOGIES, INC., Saratoga, CA (US)

(21) Appl. No.: 12/843,572

(22) Filed: Jul. 26, 2010

(51) Int. Cl. A61M 25/04

(52) U.S. Cl. 604/175; 604/506

(57) ABSTRACT

A device for use in a process that involves accessing a bodily space includes a flexible tube having a proximal end, a distal end, and a channel extending between the proximal and distal ends of the flexible tube, and a tissue engagement device coupled to the distal end of the flexible tube, wherein the tissue engagement device comprises a body with threads and a port located on the body, the port being in fluid communication with the channel of the flexible tube.

### GOVERNMENT'S RIGHTS

This invention was made with Government support under Grant No. 0848916 awarded by the National Science Foundation. The Government has certain rights in this invention.

### FIELD

The field of the application relates to devices and methods for accessing a space in a body, and more particularly, for devices and methods for accessing epidural space.

### BACKGROUND

Epidural anesthesia blocks pain sensation at nerve roots that branch directly from the spinal cord by bathing them with local anesthetic agents or opioids delivered to the epidural space, a small region adjacent to the outer protective covering of the spinal cord. This route of drug delivery provides an effective method for pain control during childbirth, major surgery, and chronic back pain as well as many other types of pain that may be debilitating in nature or affect a patient's quality of life.

Accessing the epidural space to administer anesthetic remains challenging due to its small size and proximity to the spinal cord and risk of complications, such as dural puncture, epidural vein puncture, and failure of pain control. Existing technique for accessing the epidural space involves using a sharp needle to pierce through the ligamentum flavum. Once the distal tip of the needle reaches the epidural space, a separate catheter is then inserted through the needle to reach the epidural space, thereby allowing fluid to be delivered to the epidural space using the catheter. Such technique can lead to inadvertent puncture of the dura or damage to the tissue in the area of the spinal column as well as failure of pain control. In addition, inserting a separate catheter through the needle may lead to bending or kinking of the catheter, is inconvenient, and may complicate the process. Furthermore, such existing technique requires significant training to proficiency.

For the foregoing reasons, Applicants of the subject application determine that it would be desirable to have a new device and method for accessing the epidural space.

### SUMMARY

In accordance with some embodiments, a device for use in a process that involves accessing a bodily space includes a shaft having a proximal end, a distal end, and a lumen extending between the proximal and distal ends of the shaft, a flexible tube having a proximal end, a distal end, and a channel extending between the proximal and distal ends of the flexible tube, wherein at least a part of the tube is located in the lumen of the shaft, and a tissue engagement device coupled to the distal end of the flexible tube, wherein the shaft is detachably coupled to the tissue engagement device.

In accordance with other embodiments, a device for use in a process that involves accessing a bodily space includes a flexible tube having a proximal end, a distal end, and a channel extending between the proximal and distal ends of the flexible tube, and a tissue engagement device coupled to the distal end of the flexible tube, wherein the tissue engagement device comprises a body with threads and a port located on the body, the port being in fluid communication with the channel of the flexible tube.

In accordance with other embodiments, a method for use in a process that involves accessing a bodily space includes inserting a tissue engagement element into a body, the tissue engagement element coupled to a shaft, operating the shaft to cause the tissue engagement element to engage with tissue, decoupling the shaft from the tissue engagement element, pulling the shaft proximally to expose a flexible tube inside the body, and using the flexible tube to deliver fluid or an object into the body.

In accordance with other embodiments, a method for use in a process that involves accessing a bodily space includes inserting a tissue engagement element into a body, the tissue engagement element coupled to a flexible tube, inserting the tissue engagement element at least partially into ligamentum flavum tissue to anchor the tissue engagement element against the ligamentum flavum tissue, and using the flexible tube to deliver fluid or an object into the body.

Other and further aspects and features will be evident from reading the following detailed description of the embodiments, which are intended to illustrate, not limit, the invention.

### DESCRIPTION OF THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments and are not therefore to be considered limiting of its scope.

FIG. 1 illustrates a device for accessing an epidural space in accordance with some embodiments.

FIG. 2 illustrates a distal end of a component of the device of FIG. 1 in accordance with some embodiments.

FIG. 3A illustrates a distal end of a component of the device of FIG. 1 in accordance with some embodiments.

FIG. 3B illustrates a distal end of a component of the device of FIG. 1 in accordance with other embodiments.

FIG. 4 illustrates a proximal end of a component of the device of FIG. 1 in accordance with some embodiments.

FIGS. 5A-5F illustrate a process of using the device of FIG. 1 in accordance with some embodiments.

FIG. 6 illustrates a distal end of a component of the device of FIG. 1 in accordance with other embodiments.

FIG. 7 illustrates another device for accessing an epidural space in accordance with other embodiments.

### DETAILED DESCRIPTION OF THE EMBODIMENTS

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

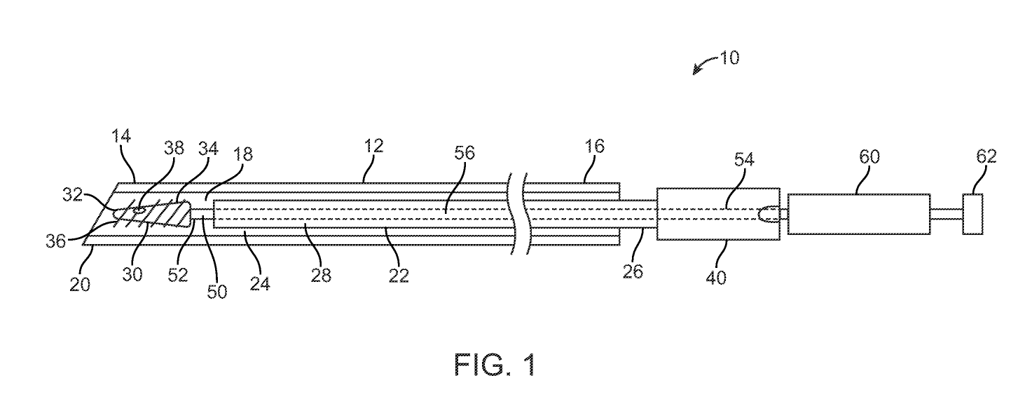

FIG. 1 illustrates a device 10 for accessing an epidural space in accordance with some embodiments. The device 10 includes an introducer sheath 12 having a distal end 14, a proximal end 16, and a lumen 18 extending between the distal end 14 and the proximal end 16. The introducer sheath 12 has a sharp distal tip 20 for allowing the introducer sheath 12 to puncture a patient's skin. The device 10 also includes a tubular shaft 22 that is housed at least partially within the lumen 18 of the introducer sheath 12. The shaft 22 has a distal end 24, a proximal end 26, and a lumen 28 extending between the distal end 24 and the proximal end 26. The device 10 further includes a tissue engagement element 30 that is configured to be detachably couple to the distal end 24 of the shaft 22, and a handle 40 coupled to the proximal end 26 of the shaft 22. As shown in the illustrated embodiments, the device 10 also includes a flexible tube 50 having a distal end 52 connected to the tissue engagement element 30, a proximal end 54 that is configured to be coupled to a fluid source 60, and a lumen 56 extending between the distal end 52 and the proximal end 54. In the illustrated embodiments, the fluid source 60 is a syringe with a plunger 62. In other embodiments, the fluid source 60 may include a mechanical pump, such as an infusion machine. Also, in other embodiments, the flexible tube 50 may be for delivering other items, such as electrical lead(s), sensor(s), implantable device(s), etc.

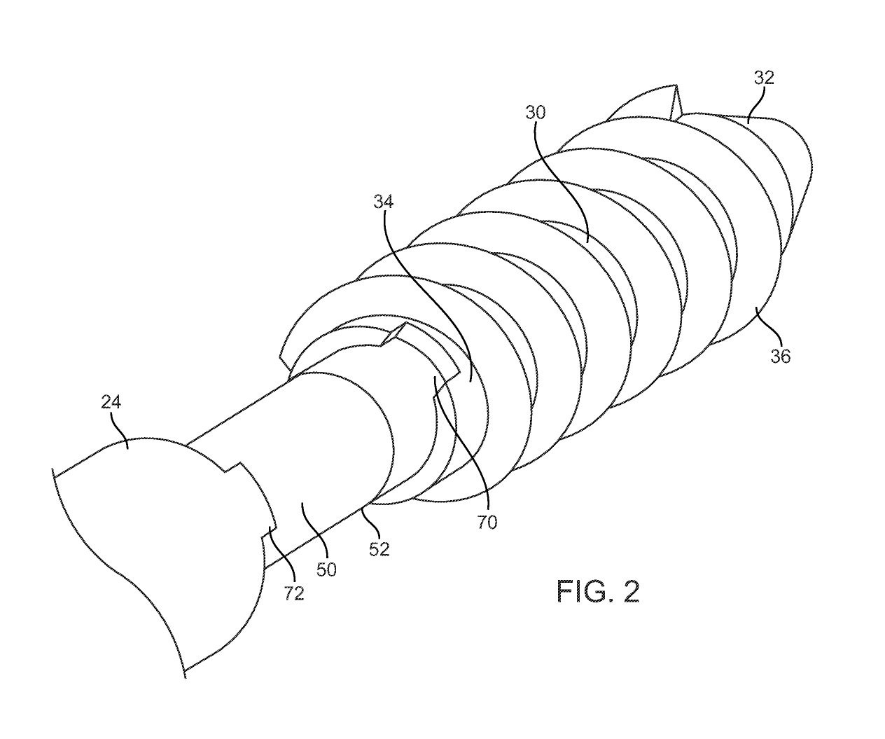

The tissue engagement element 30 has a distal end 32 with a blunt tip, and a proximal end 34 configured to detachably couple to the distal end 24 of the shaft 22. The tissue engagement element 30 also includes screw-type threads 36 configured for drilling into tissue. The tissue engagement element 30 further includes an opening 38 that is in fluid communication with the lumen 56 of the flexible tube 50. FIG. 2 illustrates the tissue engagement element 30 in further detail. As shown in the figure, the tissue engagement element 30 has a slot 70 at its proximal end 34 that is configured (e.g., sized, shaped, and positioned) for mating with a protrusion 72 at the distal end 24 of the shaft 22. Although one set of slot 70 and protrusion 72 is shown, in other embodiments, the device 10 may include multiple sets of slots 70 and protrusions 72. For example, in other embodiments, the device 10 may include two opposing slots 70 at the tissue engagement element 30, and two opposing protrusions 72 at the distal end 24 of the shaft 22. Also, in further embodiments, the slot-protrusion configuration may be reversed. For example, in other embodiments, the tissue engagement element 30 may have one or more protrusions, and the shaft 22 may have one or more slots for mating with the protrusion(s) at the tissue engagement element 30. In further embodiments, the tissue engagement element 30 and the shaft 22 may be detachably coupled to each other using other techniques, such as using a mechanical coupler.

During use, in one mode of operation, the shaft 22 may be coupled to the tissue engagement element 30 by the slot-protrusion arrangement, and the shaft 22 may be used to transmit torque applied at a proximal end to turn the tissue engagement element 30. In another mode of operation, the shaft 22 may be decoupled from the tissue engagement element 30 to expose the flexible tube 50. The method of using the device 10 will be discussed in further detail below.

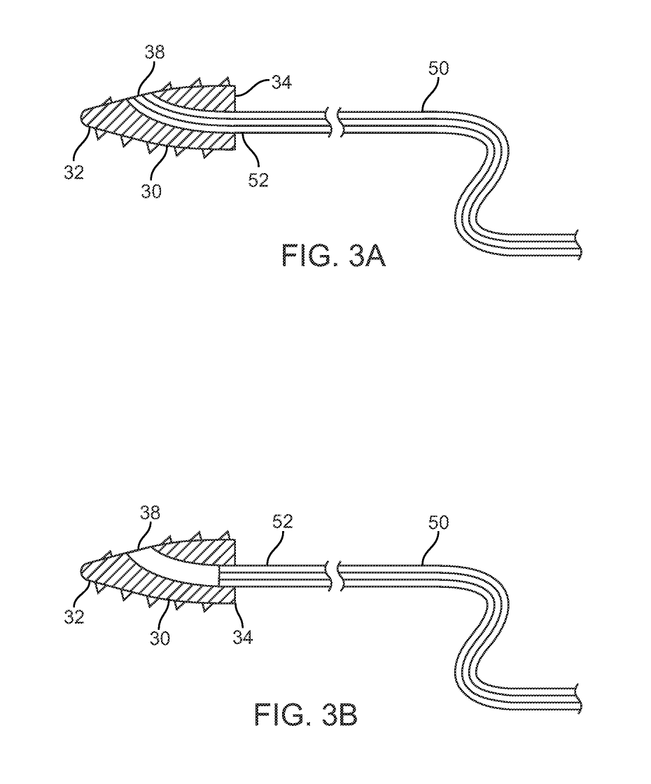

FIG. 3A illustrates a cross section of the tissue engagement element 30 in accordance with some embodiments. As shown in the figure, the distal end 52 of the flexible tube 50 extends through the tissue engagement element 30 so that it ends at the opening 38 of the tissue engagement element 30. Such configuration allows the passage at the tissue engagement element 30 to be lined by the wall of the tube 50. In other embodiments, the distal end 52 of the flexible tube 50 may not extend through the tissue engagement element 30. Instead, the distal end 52 of the flexible tube 50 may be coupled to the proximal end 34 of the tissue engagement element 30 (FIG. 3B). The tube 50 may be fixedly secured relative to the tissue engagement element 30 via glue (or a suitable adhesive) or a mechanical coupler. Alternatively, the tube 50 may be detachably secured to the tissue engagement element 30.

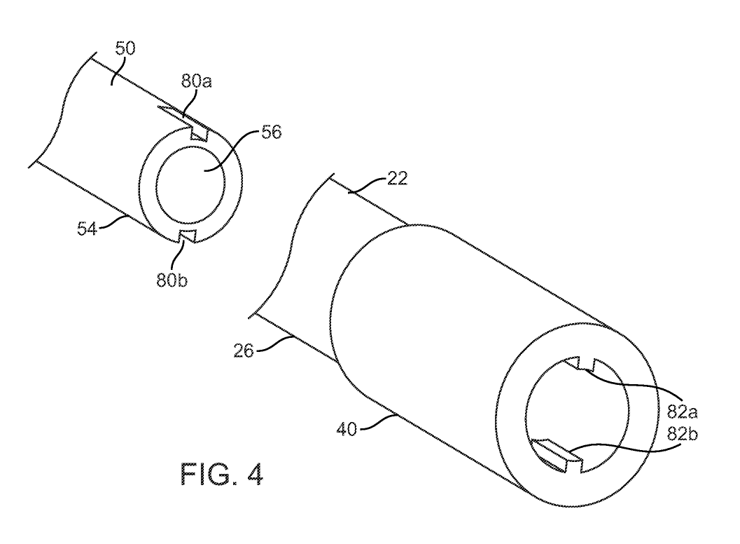

As discussed, the shaft 22 may be coupled to the tissue engagement element 30 for turning the tissue engagement element 30. In some embodiments, the proximal end 26 or the handle 40 of the shaft 22 may be detachably coupled to a proximal end 54 of the flexible tube 50 as well. Such configuration allows the flexible tube 50 to be rotated together with the shaft 22. FIG. 4 illustrates the proximal end 54 of the flexible tube 50, and the handle 40 of the shaft 22, in accordance with some embodiments. The proximal end 54 of the flexible tube 50 has a pair of slots 80a, 80b, that are configured to mate with the protrusions 82a, 82b at the inner wall of the handle 40. During use, the shaft 22 may be advanced distally to slide the protrusions 82a, 82b into the slots 80a, 80b at the tube 50. As the protrusions 82a, 82b engage with the slots 80a, 80b, the distal end 24 of the shaft 22 also mechanically engages the tissue engagement element 30 via the slot 70 and protrusion 72 arrangement. The shaft 22 may then be used to turn the tissue engagement element 30 by transferring torsional force via the mechanical coupling. In the illustrated embodiments, as the shaft 22 is being rotated, the flexible tube 50 will be turned together with the shaft 22 because the protrusion 82 is engaged with the slot 80. Such configuration may be desirable because it prevents the flexible tube 50 from twisting relative to the shaft 22 as the shaft 22 is being rotated.

In the above embodiments, the protrusions 82a, 82b are illustrated as being at the inner wall of the handle 40. In other embodiments, the protrusions 82a, 82b may be at the inner wall of the shaft 22 (e.g., at the proximal end 26 of the shaft 22).

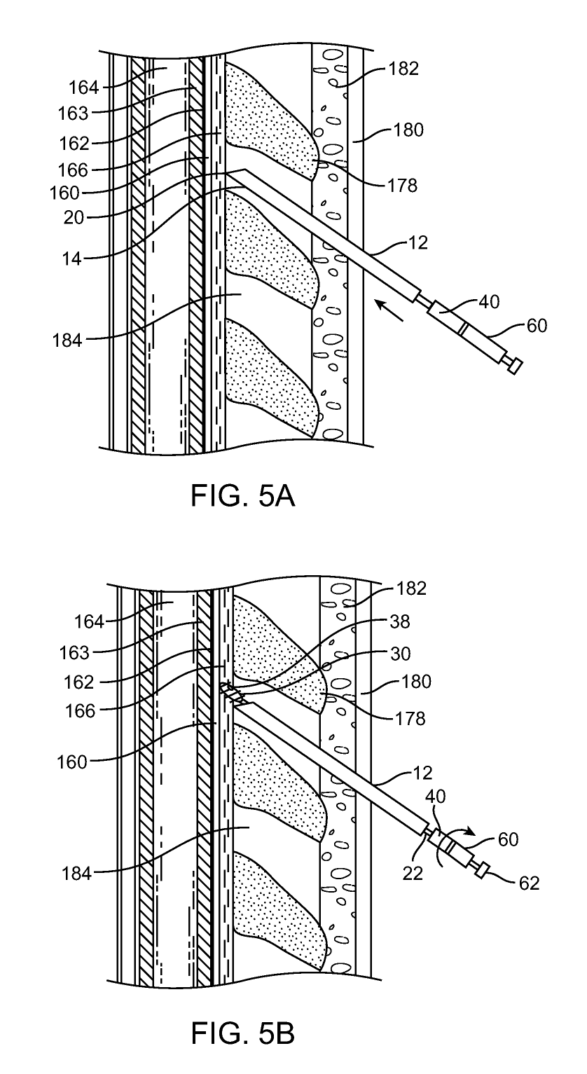

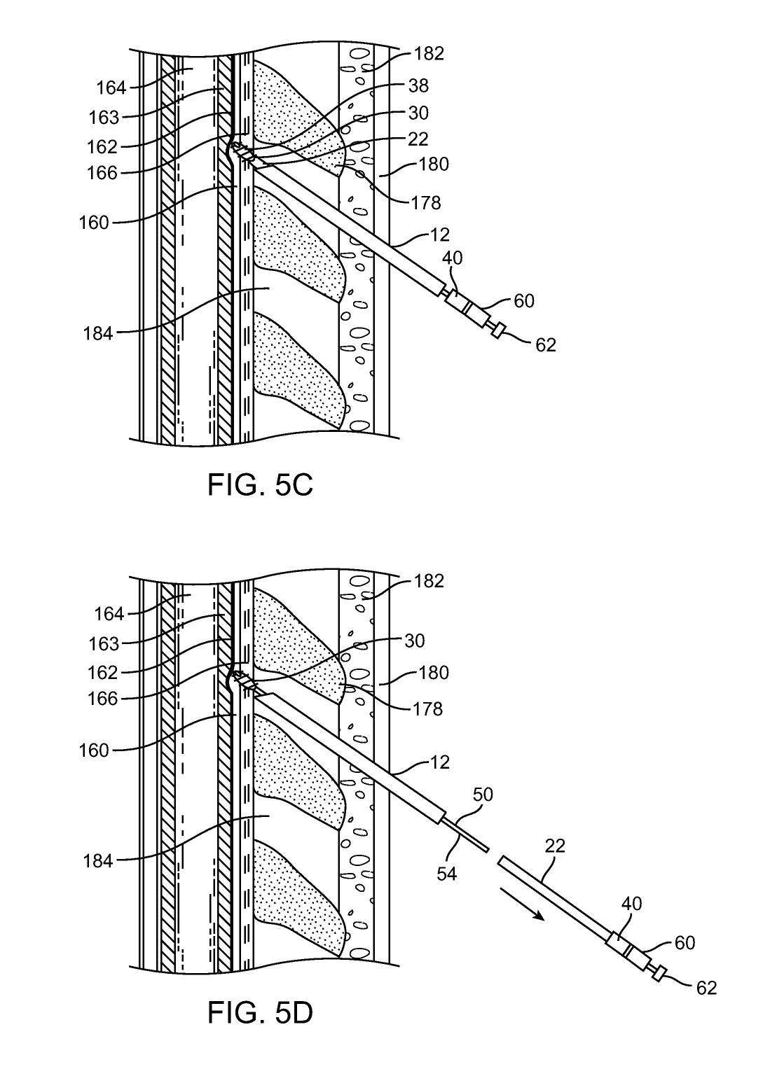

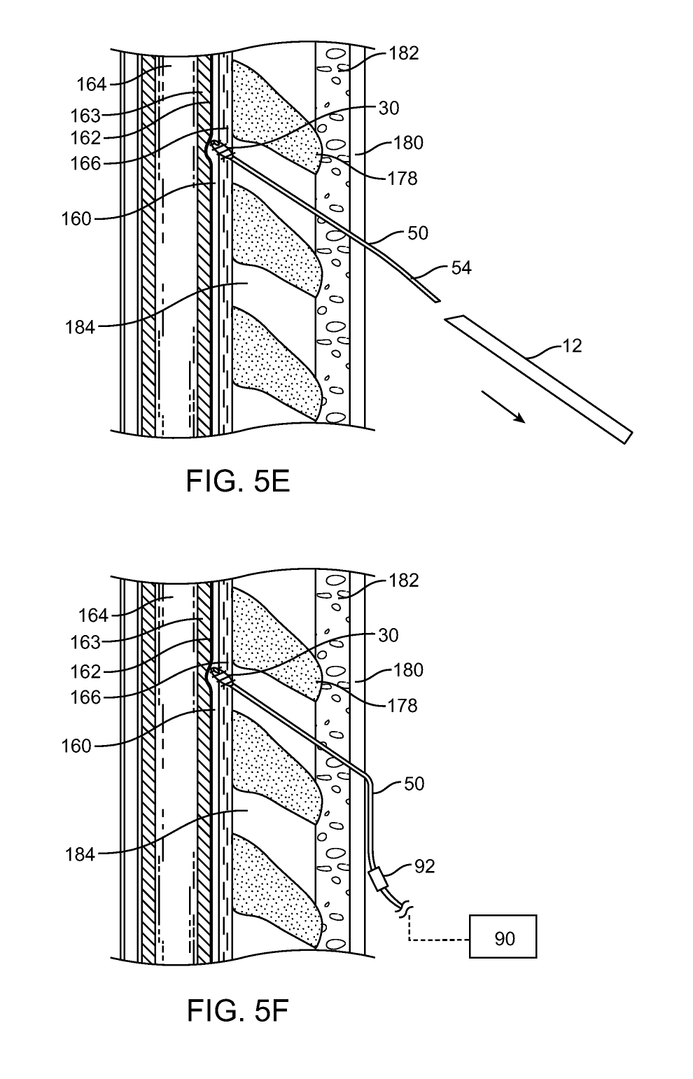

FIGS. 5A-5F illustrate a method of using the device 10 of FIG. 1 to access an epidural space in accordance with some embodiments. The epidural space 160 is a potential space that is initially in a collapsed configuration, and is enlarged when the tissues that bound it are separated. As shown in FIG. 5A, the tissues that define, or are adjacent to, the epidural space 160 including the dura mater (or dura 162) which is a protective covering that sheaths the spinal cord 164, the ligamentum flavum 166 which is a ligament adjacent to the dura 162 that runs longitudinally along the spinal column, cerebral spinal fluid (CSF) 163 that is between the dura 162 and the spinal cord 164, and the bony sides of the vertebral canal.

To access the epidural space 160, the patient is positioned either seated or on his/her side, and is instructed to flex the back outward to maximize the spacing between the outer vertebral components. The spinal processes 178 are palpated, and the interlaminar space is estimated. The doctor then uses the introducer sheath 12, with the tissue engagement element 30 housed within it, to puncture the patient's skin 180 (FIG. 5A). Alternatively, instead of housing the tissue engagement element 30 within the introducer sheath 12, another device, such as a stylet, may be housed within the introducer sheath 12. The advantage of having the tissue engagement element 30 (or another device) inside the introducer sheath 12 while the introducer sheath 12 is used to cut (e.g., pierce) into tissue is that it may prevent the introducer sheath 12 from coring tissue. The introducer sheath 12 is then advanced distally to cut (e.g., pierce through) soft tissue 82 and the surrounding tissue 184 that is between two spinal processes 178. The introducer sheath 12 is advanced distally until the distal tip 20 contacts the ligamentum flavum 166. When the distal tip 20 contacts the ligamentum flavum 166, the doctor holding the device 10 will sense a higher resistance (as the ligamentum flavum 166 is more dense than the less dense surrounding tissue 184), thereby knowing that the distal end 14 of the introducer sheath 12 has been desirably positioned.

Next, the doctor then advances the handle 40 distally to push the tissue engagement element 30 out of the distal opening at the introducer sheath 12, and the doctor then turns the handle 40 to rotate the tissue engagement element 30, thereby drilling the tissue engagement element 30 into the ligamentum flavum 166 (FIG. 5B). During this process, the shaft 22 is mechanically coupled to the tissue engagement element 30 such that torsional force applied at the handle 40 can be transmitted to the tissue engagement element 30. Also, the fluid source 60 is coupled to the handle 40, so that the flexible tube 50 within the shaft 12 is in fluid communication with the fluid source 60. Because the proximal end 54 of the tube 50 is coupled to the shaft 22, turning of the shaft 22 will cause the tube 50 to rotate with the shaft 22 relative to the fluid source 60. However, the interface between the fluid source 60 and the tube 50 is configured such that a seal is formed therebetween while allowing the tube 50 to be rotated relative to the fluid source 60.

As the doctor rotates the handle 40, the doctor also applies pressure at the plunger 62 of the fluid source 60. While the tissue engagement element 30 is being advanced through the ligamentum flavum 166, the opening 38 is inside the layer of the ligamentum flavum 166, which prevents fluid from being delivered out of the opening 38. Thus, the doctor will sense a relatively high pressure at the plunger 62 while the tissue engagement element 30 is being drilled through the ligamentum flavum 166. Alternatively, a pressure application system may be used to automatically apply the pressure while the tissue engagement element 30 is being drilled through the ligamentum flavum 166. In such case, the pressure application system may include a pressure for sensing the pressure that is being applied.

As shown in FIG. 5C, the doctor continues to rotate the handle 40 to drill the tissue engagement element 30 distally, until the opening 38 at the tissue engagement element 30 exits the ligamentum flavum 166. At the position shown in the figure, the tissue engagement element 30 is anchored against the ligamentum flavum 166 due to the threads 36 on the tissue engagement element 30. Thus, the threads 36 of the tissue engagement element 30 allow the tissue engagement element 30 to advance relative to the ligamentum flavum 166 via turning action, and also allows the tissue engagement element 30 to be anchored against the tissue next to the threads 36. As shown in the figure, as soon as the opening 38 exits the layer of ligamentum flavum 166, fluid at the fluid source 60 may be delivered out of the opening 38, and thus, the doctor will sense a decrease of pressure at the plunger 62. When the doctor senses that there is a decrease of pressure at the plunger 62, the doctor then knows that the tissue engagement element 30 has been desirably placed—i.e., that the opening 38 of the tissue engagement element 30 has reached the epidural space 160. In other embodiments, if a pressure application system is used to apply the pressure, the sensor of the pressure application system will sense the decrease of pressure. In some cases, the pressure application system further includes a signal generator for generating a signal, such as an audio signal, a visual signal, or combination of both. When the sensor senses that there is a drop in the application of pressure, the signal generator will generate the signal to let the user know that the tissue engagement element 30 has been desirably placed. Using the tissue engagement element 30 with the screw threads 36 to penetrate the ligamentum flavum 166 is advantageous because it allows the doctor to slowly and controllably advance the distal opening 38 of the device 10 into the epidural space 160 in a safe manner.

Next, the shaft 22 is decoupled from the tissue engagement element 30 (e.g., by pulling proximally relative to the tissue engagement element 30), and the shaft 22 is removed from the rest of the device 10 (FIG. 5D). The introducer sheath 12 is then removed from the patient by pulling the introducer sheath 12 proximally (FIG. 5E), thereby exposing the flexible tube 50 inside the patient. In other embodiments, the shaft 22 and the introducer sheath 12 may be removed together at the same time. The proximal end 54 of the flexible tube 50 is then coupled to another fluid source 90 via a mechanical coupler 92 (FIG. 5F). In the illustrated embodiments, the fluid source 90 may include pain medication (such as an infusion machine) for delivering pain medication to the patient. In other embodiments, the fluid source 90 may be configured to deliver other substance, such as saline, agent, drug, etc., to the patient. In further embodiments, the flexible tube 50 may be used to deliver a device/instrument, such as electrical lead(s), sensor(s), implantable device(s), etc., into the patient.

As illustrated in the above embodiments, having the flexible tube 50 built into the device 10 is advantageous because it obviates the need to have a separate delivery catheter, and it obviates the need to have the extra step of inserting the delivery catheter through the shaft 22 to reach the opening 38 of the tissue engagement element 30. This, in turn, eliminates the risk of bending and/or kinking of the flexible tube, misplacement of the catheter outside of the epidural space, and risk of infection (because one less piece of equipment is needed to be inserted into the patient). Also, as illustrated in the above embodiments, devices and methods described herein facilitate controlled entry into the epidural space 160 by controllably advancing the tissue engagement element 30 into the ligamentum flavum 166. This is advantageous because it reduces the risk of injuring the dura. Also, the blunt distal tip at the tissue engagement element 30 is advantageous because it further reduces the risk of injuring the dura.

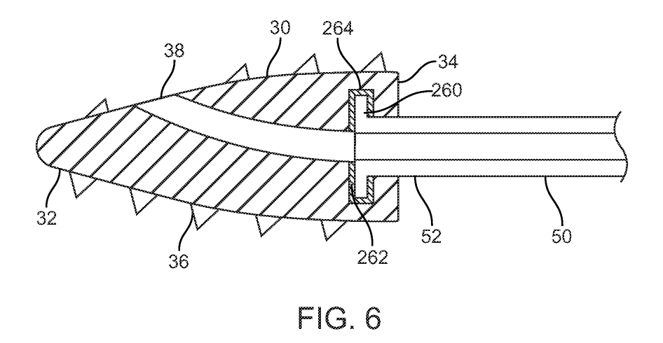

In the above embodiments, the flexible tube 50 is described as being fixedly secured to the tissue engagement element 30. In other embodiments, the flexible tube 50 may not be fixedly secured to the tissue engagement element 30. Instead, the flexible tube 50 may be rotatably coupled to the tissue engagement element 30. FIG. 6 illustrates a variation of the device 10 in which the flexible tube 50 is rotatably coupled to the tissue engagement element 30. As shown in the figure, the distal end 52 of the tube 50 has a flange 260 that is configured (e.g., sized and shaped) to be placed in a mating groove 262 at an inner surface of the tissue engagement element 30. A seal 264 may be optionally provided to seal between the flange 260 and the groove 262. The illustrated coupling configuration allows the tissue engagement element 30 to be rotatable relative to the flexible tube 50. In such cases, the proximal end 54 of the tube 50 does not need to be mechanically coupled to the handle 40 or to the proximal end 26 of the shaft 22 (like that shown in FIG. 4). Instead, as the handle 40 is rotated relative to the fluid source 60, the tube 50 (which is coupled to the fluid source 60) will stay in place relative to the fluid source 60, while the tissue engagement element 30 is rotated relative to the tube 50 by the shaft 22.

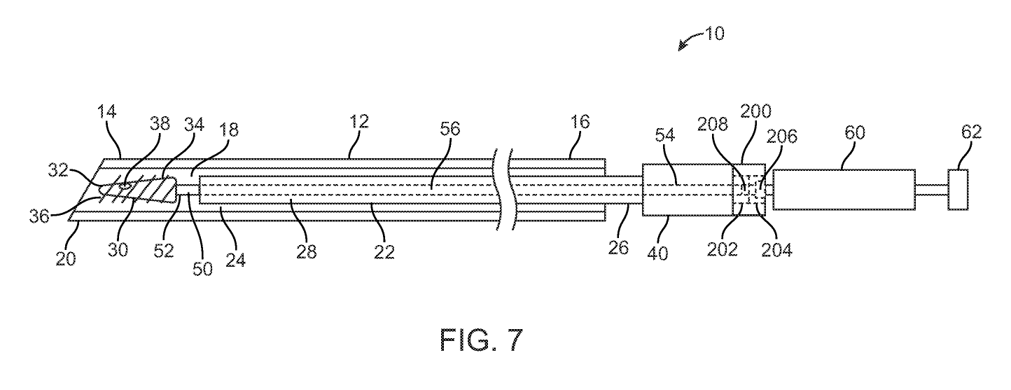

In further embodiments, the device 10 may optionally include a coupler 200 for coupling the shaft 22 and the flexible tube 50 to the fluid source 60 (FIG. 7). The coupler 200 includes a first portion 202 that is rotatably coupled to a second portion 204. The first portion 202 has an opening 206 for coupling to the fluid source 60 (or another fluid source), and the second portion 204 has an opening 208 for coupling to the flexible tube 50. The first and second portions 202, 204 abut against each other such that they form a seal therebetween. Because the first and second portions 202, 204 are rotatable relative to each other, the tube 50 that is coupled to the second portion 504 may be rotated relative to the fluid source 60 (that is coupled to the first portion 502), while the shaft 22 is rotated relative to the fluid source 60 to advance the tissue engagement element 30. In other embodiments, the coupler 200 may optionally include a seal (e.g., an O-ring seal) disposed between the first and second portions 202, 204.

It should be noted that the configuration of the device 10 is not limited to the examples described herein, and that the device 10 may have other configurations in other embodiments. For example, in other embodiments, the flexible tube 50 may be configured (e.g., sized and/or shaped) to slidably couple to the tissue engagement element 30. In one implementation, the distal portion of the flexible tube 50 may be configured to have a frictional fit against an inner wall of the tissue engagement element 30. Such configuration allows the flexible tube 50 to couple to the tissue engagement element 30, while also allowing the flexible tube 50 to translate axially (e.g., distally or proximally) relative to the tissue engagement element 30. During use, after the tissue engagement element 30 has been anchored relative to the ligamentum flavum 166, the flexible tube 50 may be advanced distally so that its distal end 52 exits out of the opening 38 at the tissue engagement element 30. The flexible tube 50 may then be used to deliver substance, such as pain medication, agent, drug, etc., into the body. In further embodiments, the inner wall of the tissue engagement element 30 may have one or more protrusions for mating with respective one or more elongated grooves on the exterior surface of the flexible tube 50. The groove(s) extend longitudinally along at least a portion of a length of the tube 50, thereby allowing the flexible tube 50 to be slidably coupled to the tissue engagement element 30. In alternative embodiments, the protrusion(s) may be on the exterior surface of the flexible tube 50, and the groove(s) may be defined on the inner wall of the tissue engagement element 30.

In addition, in further embodiments, the tissue engagement element 30 does not need to have the configuration described previously, and the tissue engagement element 30 may have any configuration as long as it can be inserted into ligamentum flavum tissue 166, and/or anchor itself against the ligamentum flavum 166.

Also, in other embodiments, the device 10 may not include the handle 40. Instead, the proximal portion of the shaft 22 may be used as a handle. In addition, in other embodiments, the device 10 may further include a handle coupled to the proximal end 16 of the introducer sheath 12. In further embodiments, the device 10 may not include the introducer sheath 12. In such cases, the tissue engagement element 30 and the shaft 22 may be used to puncture the patient's skin to access the epidural space 160. In addition, in other embodiments, the proximal end 54 of the flexible tube 50 may include two ports, wherein one port is for fluid communication with the fluid source 60 (e.g., a syringe carrying saline) for use during advancement of the tissue engagement element 30, and the other port is for fluid communication with another fluid source (e.g., fluid source 90) for delivering fluid, such as pain medication, to the patient after the tissue engagement element 30 has been anchored against the ligamentum flavum 166. In such cases, the proximal end 54 of the flexible tube 50 may include a valve control for selectively allowing fluid flow to the distal end 52 from either one of the two fluid sources.

A person skilled in the art will appreciate the foregoing as only illustrative of the various embodiments, and that various modifications may be made to both the epidural space accessing devices and the methods presented without departing from the scope and spirit of the invention.

1. A device for use in a process that involves accessing a bodily space, comprising:
a shaft having a proximal end, a distal end, and a lumen extending between the proximal and distal ends of the shaft;
a flexible tube having a proximal end, a distal end, and a channel extending between the proximal and distal ends of the flexible tube, wherein at least a part of the tube is located in the lumen of the shaft; and
a tissue engagement device coupled to the distal end of the flexible tube;
wherein the shaft is detachably coupled to the tissue engagement device.

2. The device of claim 1, wherein the distal end of the shaft is detachably coupled to a proximal end of the tissue engagement device.

3. The device of claim 2, wherein the proximal end of the tissue engagement device comprises a mechanism for detachably coupling to the distal end of the shaft, the mechanism selected from the group consisting of a recess, a protrusion, and screw threads.

4. The device of claim 1, wherein the shaft is configured for transferring torque to the tissue engagement device when the tissue engagement device is coupled to the shaft.

5. The device of claim 1, wherein when the shaft is decoupled from the tissue engagement device, the shaft can be positioned proximally to expose the at least a part of the flexible tube.

6. The device of claim 1, wherein the tissue engagement device comprises a port in fluid communication with the channel of the flexible tube.

7. The device of claim 1, wherein the tissue engagement device comprises a body having a proximal end, a distal end, and threads located between the proximal and distal ends.

8. The device of claim 7, wherein the tissue engagement device further comprises a port located between the proximal and distal ends of the body, the port being in fluid communication with the channel of the flexible tube.

9. The device of claim 1, wherein the proximal end of the flexible tube is configured for coupling to a fluid source.

10. The device of claim 9, further comprising the fluid source, wherein the fluid source comprises pain medication.

11. The method of claim 1, wherein the tissue engagement device is configured to anchor itself to a ligamentum flavum.

12. A device for use in a process that involves accessing a bodily space, comprising:
a flexible tube having a proximal end, a distal end, and a channel extending between the proximal and distal ends of the flexible tube; and
a tissue engagement device coupled to the distal end of the flexible tube;
wherein the tissue engagement device comprises a body with threads and a port located on the body, the port being in fluid communication with the channel of the flexible tube.

13. The device of claim 12, wherein the tissue engagement device comprises a blunt distal tip.

14. The device of claim 12, wherein the proximal end of the flexible tube is configured for coupling to a fluid source.

15. The device of claim 14, further comprising the fluid source, wherein the fluid source comprises pain medication.

16. The device of claim 12, wherein the tissue engagement device comprises a mechanism for detachably coupling the tissue engagement device to a shaft.

17. The device of claim 16, wherein the mechanism comprises a recess for mating with a protrusion at a distal end of the shaft.

18. The device of claim 16, wherein the mechanism comprises a protrusion for mating with a recess at a distal end of the shaft.

19. The device of claim 16, wherein the mechanism comprises screw threads.

20. The device of claim 12, further comprising a shaft that is detachably coupled to the tissue engagement device, wherein the shaft is configured for transferring torque to the tissue engagement device when the tissue engagement device is coupled to the shaft.

21. The device of claim 20, wherein when the shaft is decoupled from the tissue engagement device, the shaft can be positioned proximally to expose the at least a part of the flexible tube.

22. The device of claim 12, wherein the tissue engagement device is configured to anchor itself to a ligamentum flavum.

23. A method for use in a process that involves accessing a bodily space, comprising:
inserting a tissue engagement element into a body, the tissue engagement element coupled to a shaft;
operating the shaft to cause the tissue engagement element to engage with tissue;
decoupling the shaft from the tissue engagement element;
pulling the shaft proximally to expose a flexible tube inside the body; and
using the flexible tube to deliver fluid or an object into the body.

24. The method of claim 23, wherein the fluid comprises a pain medication, and the flexible tube is used to deliver the pain medication into an epidural space in the body.

25. The method of claim 23, wherein the tissue comprises ligamentum flavum tissue.

26. The method of claim 23, wherein the act of operating the shaft comprises rotating the shaft to screw the tissue engagement element into the tissue.

27. The method of claim 26, wherein the act of rotating the shaft is accomplished by rotating a handle that is coupled to the shaft.

28. The method of claim 26, wherein the tissue engagement element comprises threads for anchoring the tissue engagement element against the tissue.

29. The method of claim 23, wherein the tissue engagement element comprises an opening in fluid communication with a lumen in the flexible tube, and wherein the fluid or the object is delivered from the lumen in the flexible tube through the opening at the tissue engagement element into the body.

30. The method of claim 23, wherein the object comprises an instrument.

31. A method for use in a process that involves accessing a bodily space, comprising:
placing a tissue engagement element into a body, the tissue engagement element coupled to a flexible tube;
inserting the tissue engagement element at least partially into ligamentum flavum tissue to anchor the tissue engagement element against the ligamentum flavum tissue; and
using the flexible tube to deliver fluid or an object into the body.

32. The method of claim 31, wherein the fluid comprises a pain medication, and the flexible tube is used to deliver the pain medication into an epidural space in the body.

33. The method of claim 31, wherein the act of inserting the tissue engagement element comprises rotating the flexible tube to screw the tissue engagement element into the ligamentum flavum tissue.

34. The method of claim 31, wherein the tissue engagement element comprises threads for anchoring the tissue engagement element against the ligamentum flavum tissue.

35. The method of claim 31, wherein the tissue engagement element comprises an opening in fluid communication with a lumen in the flexible tube, and wherein the fluid or the object is delivered from the lumen in the flexible tube through the opening at the tissue engagement element into the body.

36. The method of claim 31, wherein the object comprises an instrument.
